(19) United States
(12) Patent Application Publication
(10) Pub. No.: US 2002/0049152 A1
(43) Pub. Date: Apr. 25, 2002

(54) Methods for immobilizing polypeptides

(75) Inventors: Steffen Nock, Redwood City, CA (US); Jens Sydor, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Zyomyx, Inc., Hayward, CA

(21) Appl. No.: 09/884,269

(22) Filed: Jun. 19, 2001

Related U.S. Application Data
(60) Provisional application No. 60/212,620, filed on Jun. 19, 2000.

(51) Int. Cl. A61K 38/00; A61K 31/70

(52) U.S. Cl. 514/2; 514/25

(57) ABSTRACT

This invention provides methods for immobilizing polypeptides, for forming arrays of polypeptides arranged on a support, and arrays produced using the methods of the invention. The immobilized polypeptides of the invention are generally in the same orientation, can be full-length and biologically active, and can be readily screened for a desired activity.

### CROSS-REFERENCES TO RELATED APPLICATIONS

This application claims the benefit of U.S. Provisional Patent application Serial No. 60/212,620, filed on Jun. 19, 2000, which is herein incorporated by reference in its entirety.

### BACKGROUND OF THE INVENTION

1. Field of the Invention

This invention pertains to the field of immobilizing a polypeptide to a surface, and methods of using such immobilized polypeptides for proteomics and high-throughput screening.

2. Background

A vast number of new drug targets are now being identified using a combination of genomics, bioinformatics, genetics, and high-throughput biochemistry. Genomics provides information on the genetic composition and the activity of an organism's genes. Bioinformatics uses computer algorithms to recognize and predict structural patterns in DNA and proteins, defining families of related genes and proteins. Genomics, however, cannot provide a complete understanding of the cellular processes that are involved in disease processes because such processes are mediated by proteins. Genomics alone provides little or no information as to, for example, the relative abundance of different proteins in a cell, and the types of post-translational modifications present on proteins.

Proteomics is providing a new weapon for bridging the gap between genomics and disease processes. Proteomics involves the study of proteins in biological samples. For example, proteomics can involve comparing the proteins present in a diseased cell to those in a non-diseased cell to identify disease-specific proteins. The combination of proteomics with the other approaches is expected to greatly boost the number of potential drug targets that are of interest for the development of new drugs.

The number of chemical compounds available for screening as potential drugs is also growing dramatically due to recent advances in combinatorial chemistry, the production of large numbers of organic compounds through rapid parallel and automated synthesis. The compounds produced in the combinatorial libraries being generated will far outnumber those compounds being prepared by traditional, manual means, natural product extracts, or those in the historical compound files of large pharmaceutical companies. Both the rapid increase of new drug targets and the availability of vast libraries of chemical compounds creates an enormous demand for new technologies which improve the screening process.

The complexity of drug screening is further complicated by the need to identify highly specific lead compounds early in the drug discovery process. Proteins within a structural family share similar binding sites and catalytic mechanisms. Often, a compound that effectively interferes with the activity of one family member, as desired, but also interferes with other members of the same family. Cross-reactivity of a drug with related proteins can be the cause of low efficacy or even side effects in patients. For instance, AZT, a major treatment for AIDS, blocks not only viral polymerases, but also human polymerases, causing deleterious side effects. Cross-reactivity with closely related proteins is also a problem with nonsteroidal anti-inflammatory drugs (NSAIDs) and aspirin. These drugs inhibit cyclooxygenase-2, an enzyme which promotes pain and inflammation. However, the same drugs also strongly inhibit a related enzyme, cyclooxygenase-1, that is responsible for keeping the stomach lining and kidneys healthy, leading to common side-effects including stomach irritation. Using standard technology to discover such additional interactions requires a tremendous effort in time and costs and as a consequence is simply not done. The ability to analyze a multitude of members of a protein family or forms of a polymorphic protein in parallel (multitarget screening) would enable quick identification of highly specific lead compounds that do not exhibit undesirable cross-reactivity.

Current technological approaches for obtaining high-throughput screening of proteins and other targets for drugs include multiwell-plate based screening systems, cell-based screening systems, microfluidics-based screening systems, and screening of soluble targets against solid-phase synthesized drug components. For example, methods are available for synthesizing potential drugs on a solid phase and assaying the immobilized drugs for ability to interact with a soluble protein or other target. However, screening of soluble targets against solid-phase synthesized drug components is intrinsically limited. The surfaces required for solid state organic synthesis are chemically diverse and often cause the inactivation or non-specific binding of proteins, leading to a high rate of false-positive results. Furthermore, the chemical diversity of drug compounds is limited by the combinatorial synthesis approach that is used to generate the compounds at the interface. Another major disadvantage of this approach stems from the limited accessibility of the binding site of the soluble target protein to the immobilized drug candidates.

Attachment of the drug target, rather than the potential drug, to a solid support has proven useful for screening of molecules that interact with DNA. Miniaturized DNA chip technologies have been developed (for example, see U.S. Pat. Nos. 5,412,087, 5,445,934 and 5,744,305) and are currently being exploited for nucleic acid hybridization and other assays. However, DNA biochip technology is not transferable to protein arrays because the chemistries and materials used for DNA biochips are not readily transferable to use with proteins. Nucleic acids withstand temperatures up to 100° C., can be dried and re-hydrated without loss of activity, and can be bound directly to organic adhesion layers supported by materials such as glass while maintaining their activity. In contrast, proteins must remain hydrated, kept at ambient temperatures, and are very sensitive to the physical and chemical properties of the support materials. Therefore, maintaining protein activity at the liquid-solid interface requires entirely different immobilization strategies than those used for nucleic acids. Additionally, the proper orientation of the protein at the interface is desirable to ensure accessibility of their active sites with interacting molecules. With miniaturization of the chip and decreased feature sizes the ratio of accessible to non-accessible proteins becomes increasingly relevant and important.

For the foregoing reasons, there is a need for miniaturized protein arrays, and for methods of synthesizing such arrays. The present invention fulfills these and other needs.

### SUMMARY OF THE INVENTION

In one aspect the present invention provides for methods for immobilizing a polypeptide to a surface. These methods comprise contacting a polypeptide which comprises an ester or thioester, with an anchor molecule comprising a first nucleophilic group at a 2 or 3 position relative to a second nucleophilic group, wherein the ester or thioester undergoes a trans-esterification reaction with the first nucleophilic group, thus forming an intermediate compound in which the polypeptide is attached to the anchor molecule through the first nucleophilic group; and attaching the anchor molecule to a surface.

In some embodiments, the polypeptide comprising an ester or a thioester is obtained by use of inteins. These methods generally involve expressing a chimeric gene that encodes a fusion protein which comprises: a) the polypeptide, and b) an intein, or a functional portion thereof, which is joined to the polypeptide at a splice junction at the amino terminus of the intein. The carboxyl terminus of the intein generally lacks a functional splice junction. The fusion protein is contacted with a nucleophilic compound which releases the polypeptide from the intein at the splice junction and forms the polypeptide that comprises a terminal ester or thioester.

The present invention provides methods for forming an array of immobilized polypeptides. The arrays are composed of a plurality of polypeptide species attached to a surface. The methods involve contacting members of a population of polypeptide species, each of which comprises an ester or thioester, with anchor molecules that have a first nucleophilic group at a 2 or 3 position relative to a second nucleophilic group. The ester or thioester undergoes a trans-esterification reaction with the first nucleophilic group, thus forming an intermediate compound in which the polypeptides are attached to the anchor molecules through the first nucleophilic group. The intermediate compound can then undergo an intramolecular rearrangement in which the second nucleophilic group on the anchor molecule displaces the first nucleophilic group, thus forming a more stable bond between the anchor molecule and the polypeptide (e.g., an amide bond). The anchor molecules are then attached to a surface, if not already attached prior to the linking reaction. Each polypeptide species is attached to a separate region of the surface.

Also provided are arrays of immobilized polypeptides attached to a surface. These arrays include at least a first polypeptide species and a second polypeptide species, each of which polypeptide species are: a) attached to a separate region of the surface, b) attached to the surface in the same orientation, and c) are folded in a secondary structure as required for a biological activity.

The invention also provides arrays of immobilized polypeptides attached to a surface. The surface has a plurality of surface regions, and to each surface region is attached a polypeptide species and a polynucleotide that encodes the polypeptide species.

Also provided by the invention are methods for screening a library of nucleic acids to identify a nucleic acid that encodes a polypeptide having a desired activity. These methods involve expressing a plurality of fusion proteins, each of which is encoded by an expression cassette that comprises: a) a member of the library of nucleic acids, b) an intein coding region; and c) an open reading frame that encodes a polypeptide that is displayed on a surface of a replicable genetic package. The fusion proteins are displayed on the surface of a replicable genetic package. The replicable genetic packages are then screened to identify those that display a polypeptide having the desired activity.

The invention also provides nucleic acids that include an expression cassette that has: an insertion site at which a polynucleotide can be introduced into the expression cassette, an intein coding region, and an open reading frame that encodes a polypeptide that is displayed on a surface of a replicable genetic package. In some embodiments, the carboxyl terminus of the intein coding region is mutated so that it does not function as a splice junction for intein-mediated cleavage. The introduction of a polynucleotide at the insertion site results in an open reading frame that encodes a fusion protein which comprises a polypeptide encoded by the polynucleotide, which polypeptide is attached at its carboxyl terminus to an amino terminus of the intein, and the surface-displayed polypeptide is attached to a carboxyl terminus of the intein. These expression cassettes are useful for the screening methods of the invention.

In another aspect, the invention provides for methods for immobilizing a polypeptide to a surface, wherein the method comprises contacting a polypeptide which comprises an ester or thioester, with an anchor molecule comprising a first nucleophilic group at a 2 or 3 position relative to a second nucleophilic group, wherein the ester or thioester undergoes a trans-esterification reaction with the first nucleophilic group, thus forming an intermediate compound in which the polypeptide is attached to the anchor molecule through the first nucleophilic group; wherein said intermediate compound undergoes an intramolecular rearrangement in which the second nucleophilic group on the anchor molecule displaces the first nucleophilic group, thus forming a bond between the anchor molecule and the polypeptide; and attaching the anchor molecule to a surface.

In yet another aspect, the invention provides for methods for immobilizing a polypeptide to a surface, wherein the method comprises: contacting a polypeptide which comprises an ester or thioester, with an anchor molecule comprising a reactive group selected from the group consisting of a NH₂—NH—R group and an aminooxy group wherein R represents an anchor molecule, wherein the ester or thioester reacts with the reactive group, thus forming a compound comprising a polypeptide attached to the anchor molecule through the reactive group.

In another aspect, the invention provides for a kit for use in immobilizing one or more polypeptides containing an ester or thioester to a surface of a substrate. In certain embodiments, the kit includes an anchor molecule reagent for adapting the ester or thioester containing polypeptide to the surface, the anchor molecule having a first nucleophilic group at a 2 or 3 position relative to a second nucleophilic group; wherein the ester or thioester of the one or more polypeptides undergoes a trans-esterification reaction with the first nucleophilic group, thus forming an intermediate compound in which the polypeptides are attached to the anchor molecules through the first nucleophilic group, the anchor molecule being adapted for attachment to the surface of the substrate. In other embodiments, the kit comprises an anchor molecule comprising a reactive group such as a hydrazine group (e.g., NH₂NH—R, where R is the anchor molecule), a hydroxylamine, or an aminooxy group, etc.

In some embodiments, the kits comprise a container for the contents of the kit. Certain embodiments of the kit further include, for example, a DNA vector for introducing the ester or thioester into the polypeptide, where the vector is adapted to receive a nucleic acid sequence encoding the polypeptide to form a ester or thioester polypeptide expression vector for expressing the polypeptide as an ester or thioester polypeptide having the ester or the thioester incorporated therein; where the kit further includes a chemical agent for introducing into the polypeptide an ester or thioester; where the kit further includes instructions for instructing a user to carry out methods of using the kit; where the kit further includes a substrate for attaching the anchor molecules thereto for immobilizing the polypeptides thereon; where the kit has the anchor molecule being supplied attached to the surface of the substrate for later attaching the polypeptide thereto by a user; where the kit contains said polypeptides, and where said polypeptides are supplied with said kit pre-coupled with said anchor molecule(s).

### BRIEF DESCRIPTION OF THE DRAWINGS

FIG. 1 depicts a schematic of two embodiments of methods for immobilizing a polypeptide comprising a thioester or ester to a surface. In certain embodiments, the ester or thioester is also attached to an intein. The symbol ¹R represent a reactive group such as a reactive group comprising a first nucleophilic group at a 2 or 3 position relative to a second nucleophilic group; or reactive group such as a hydrazine group, a hydroxylamine group, or an aminooxy group, etc. The structure denoted A is an anchor molecule. The symbol ²R represents a reactive group, a binding surface, amino acid residue(s), etc. on the anchor molecule that are able to bind to a surface (black bar) through covalent and/or non-covalent (e.g., ionic bonds) interactions. The symbol Y represents a sulfur or oxygen atom. In panel A, the anchor molecule comprising the reactive group ¹R is already immobilized to the surface. The reactive group ¹R then reacts with the polypeptide comprising a thioester or ester to form a polypeptide that is immobilized through the reactive group ¹R to the immobilized anchor molecule. In panel B, the polypeptide comprising the reactive group ¹R and ²R is initially free in solution. Then the reactive group ¹R reacts with the polypeptide comprising a thioester or ester to form a polypeptide that is attached to the anchor molecule through ¹R. Then this molecule is immobilized to a surface (black bar) that through covalent and/or non-covalent interactions to form a polypeptide that is immobilized to a surface through an anchor molecule containing reactive groups ¹R and attachment group ²R. The surface can be essentially be any two- or three-dimensional surface.

FIG. 2 depicts a schematic of an embodiment for immobilizing a polypeptide comprising a thioester or ester to a surface. The symbols in FIGS. 2 and 3 are the same as set out above for FIG. 1. In these embodiments, the polypeptide comprising a thioester or ester is contacted with an activating compound, as exemplified by the thiol reagent HS-R in FIG. 2. Additional activating compounds are also described herein. The activating compound displaces the intein and the resulting molecule is then contacted with the anchor molecule that is free in solution. The polypeptide is then attached to the anchor molecule through an ester or thioester bond. The anchor molecule is then affixed to the surface as set out in FIG. 1.

FIG. 3 depicts a schematic of a variant of the embodiment depicted in FIG. 2. In these embodiments, the anchor molecule is already immobilized to a surface through ²R.

### DETAILED DESCRIPTION

Definitions

A “protein” or “polypeptide” means a polymer of amino acid residues linked together by amide bonds. Typically, as used herein, the terms refer to a polymer that is of a length greater than that which is readily synthesized chemically using stepwise addition of amino acids. Thus, a “polypeptide” or “protein” generally has at least about 50 amino acids, and more preferably is at least about 60, 75, or 100 amino acids in length. A “polypeptide,” as the term is used herein, includes without limitation, a “protein,” a “polyamino acid,” a “peptide,” etc. A “polypeptide” typically has a biological activity (e.g., binding a target molecule, enzymatic activity) or other feature that is dependent upon the “polypeptide” folding into a particular secondary and/or tertiary structure. A “polypeptide” can be naturally occurring, recombinant, or synthetic, or any combination of these. A “polypeptide” can also be just a fragment of a naturally occurring “polypeptide” or peptide. A “polypeptide” can be a single molecule or can be a multi-molecular complex. The term “polypeptide” can also apply to amino acid polymers in which one or more amino acid residues is an artificial chemical analogue of a corresponding naturally occurring amino acid. An amino acid polymer in which one or more amino acid residues is an “unnatural” amino acid, not corresponding to any naturally occurring amino acid, is also encompassed by the use of the term ““polypeptide”” herein.

The term “antibody” means an immunoglobulin, whether natural or wholly or partially synthetically produced. All derivatives thereof which maintain specific binding ability are also included in the term. The term also covers any “polypeptide” having a binding domain which is homologous or largely homologous to an immunoglobulin binding domain. These “polypeptide”s can be derived from natural sources, or partly or wholly synthetically produced. An antibody can be monoclonal or polyclonal. The antibody can be a member of any immunoglobulin class, including any of the human classes: IgG, IgM, IgA, IgD, and IgE. Derivatives of the IgG class, however, are preferred in the present invention.

The term “antibody fragment” refers to any derivative of an antibody which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, and Fc fragments. The antibody fragment can be produced by any means. For instance, the antibody fragment can be enzymatically or chemically produced by fragmentation of an intact antibody or it can be recombinantly produced from a gene encoding the partial antibody sequence. Alternatively, the antibody fragment can be wholly or partially synthetically produced. The antibody fragment can optionally be a single chain antibody fragment. Alternatively, the fragment can comprise multiple chains which are linked together, for instance, by disulfide linkages. The fragment can also optionally be a multimolecular complex. A functional antibody fragment will typically comprise at least about 50 amino acids and more typically will comprise at least about 200 amino acids.

Single-chain Fvs (scFvs) are recombinant antibody fragments consisting of only the variable light chain (V_{L}) and variable heavy chain (V_{H}) covalently connected to one another by a polypeptide linker. Either V_{L} or V_{H} can be the NH₂-terminal domain. The polypeptide linker can be of variable length and composition so long as the two variable domains are bridged without serious steric interference. Typically, the linkers are comprised primarily of stretches of glycine and serine residues with some glutamic acid or lysine residues interspersed for solubility.

“Diabodies” are dimeric scFvs. The components of diabodies typically have shorter peptide linkers than most scFvs and they show a preference for associating as dimers.

An “Fv” fragment is an antibody fragment which consists of one V_{H} and one V_{L} domain held together by noncovalent interactions. The term “dsFv” is used herein to refer to an Fv with an engineered intermolecular disulfide bond to stabilize the V_{H}-V_{L} pair.

A “F(ab′)₂” fragment is an antibody fragment essentially equivalent to that obtained from immunoglobulins (typically IgG) by digestion with an enzyme pepsin at pH 4.0-4.5. The fragment can be recombinantly produced.

A “Fab′” fragment is an antibody fragment essentially equivalent to that obtained by reduction of the disulfide bridge or bridges joining the two heavy chain pieces in the F(ab′)₂ fragment. The Fab′ fragment can be recombinantly produced.

A “Fab” fragment is an antibody fragment essentially equivalent to that obtained by digestion of immunoglobulins (typically IgG) with the enzyme papain. The Fab fragment can be recombinantly produced. The heavy chain segment of the Fab fragment is the Fc piece.

An “array” is an arrangement of entities in a pattern on a substrate. Although the pattern is typically a two-dimensional pattern, the pattern can also be a three-dimensional pattern. An array of polypeptide species refers to at least two different species of polypeptide that are attached to a support. An “array” includes a plurality of microparticles, wherein each microparticle displays at least one different polypeptide as compared to another microparticle in the array. An “array” can include a plurality of replicable genetic packages.

“Microparticles” suitable for use as substrates or supports in the practice of the present invention may be selected from, according to circumstances, the group including beads, resins, and particles, used in chemical synthesis processes, isotropic and anisotropic particles, and cylinders, including stacked cylinders and/or taggants including microfiber bundles, where such particles may be made from substrate materials described elsewhere in this disclosure or known to those of ordinary skill in the art as suitable for use as a substrate as described herein, organisms and their remains such as diatoms, bacteria, spores, and yeast, where such microparticles range in size between 1 millimeters (mm) to 1 nanometers(nm), preferably from 100 micrometers (em) to 100 nm, more preferably between 10 μm to 100 nm, and are capable of being functionalized in a manner suitable for use as a substrate in the practice of the present invention.

The term “coating” means a layer that is either naturally or synthetically formed on or applied to the surface of the substrate. For instance, exposure of a substrate, such as silicon, to air results in oxidation of the exposed surface. In the case of a substrate made of silicon, a silicon oxide coating is formed on the surface upon exposure to air. In other instances, the coating is not derived from the substrate and may be placed upon the surface via mechanical, physical, electrical, or chemical means. An example of this type of coating would be a metal coating that is applied to a silicon or polymer substrate or a silicon nitride coating that is applied to a silicon substrate. Although a coating may be of any thickness, typically the coating has a thickness smaller than that of the substrate. A substrate suitable for use in the present invention may be part of a medical device, for example, a stent or appliance placed within a patient, where it is desired to have oriented display of one or more compounds from such substrate.

An “interlayer” is an additional coating or layer that is positioned between the first coating and the substrate. Multiple interlayers may optionally be used together. The primary purpose of a typical interlayer is to aid adhesion between the first coating and the substrate. One such example is the use of a titanium or chromium interlayer to help adhere a gold coating to a silicon or glass surface. However, other possible functions of an interlayer are also anticipated. For instance, some interlayers may perform a role in the detection system of the array (such as a semiconductor or metal layer between a nonconductive substrate and a nonconductive coating).

An “organic thinfilm” is a thin layer of organic molecules which has been applied to a substrate or to a coating on a substrate if present. Organic thinfilms and methods for making organic thinfilms are known in the art and include, without limitation, those described in Wagner et al. U.S. Ser. No. 09/353,555, filed Jul. 14, 1999, which is herein incorporated in its entirety for all purposes and for the purpose of teaching surface chemistries and organic thinfilms. Typically, an organic thinfilm is less than about 20 nm thick. Optionally, an organic thinfilm may be less than about 10 nm thick. An organic thinfllm may be disordered or ordered. For instance, an organic thinfilm can be amorphous (such as a chemisorbed or spin-coated polymer) or highly organized (such as a Langmuir-Blodgett film or self-assembled monolayer). An organic thinfilm may be heterogeneous or homogeneous. Organic thinfilms which are monolayers are preferred. A lipid bilayer or monolayer is a preferred organic thinfilm. Optionally, the organic thinfilm may comprise a combination of more than one form of organic thinfilm. For instance, an organic thinfilm may comprise a lipid bilayer on top of a self-assembled monolayer. A hydrogel may also compose an organic thinfilm. The organic thinfilm will typically have functionalities exposed on its surface which serve to enhance the surface conditions of a substrate or the coating on a substrate in any of a number of ways. For instance, exposed functionalities of the organic thinfilm are typically useful in the binding or covalent immobilization of the “polypeptide”s to the patches of the array. Alternatively, the organic thinfilm may bear functional groups (such as polyethylene glycol (PEG)) which reduce the non-specific binding of molecules to the surface. Other exposed functionalities serve to tether the thinfilm to the surface of the substrate or the coating. Particular functionalities of the organic thinfilm may also be designed to enable certain detection techniques to be used with the surface. Alternatively, the organic thinfilm may serve the purpose of preventing inactivation of a “polypeptide” immobilized on a patch of the array or analytes which are “polypeptide”s from occurring upon contact with the surface of a substrate or a coating on the surface of a substrate.

A “monolayer” is a single-molecule thick organic thinfilm. A monolayer may be disordered or ordered. A monolayer may optionally be a polymeric compound, such as a polynonionic polymer, a polyionic polymer, or a block-copolymer. For instance, the monolayer may be composed of a poly(amino acid) such as polylysine. A monolayer which is a self-assembled monolayer, however, is most preferred. One face of the self-assembled monolayer is typically composed of chemical functionalities on the termini of the organic molecules that are chemisorbed or physisorbed onto the surface of the substrate or, if present, the coating on the substrate. Examples of suitable functionalities of monolayers include the positively charged amino groups of poly-L-lysine for use on negatively charged surfaces and thiols for use on gold surfaces. Typically, the other face of the self-assembled monolayer is exposed and may bear any number of chemical functionalities (end groups). Preferably, the molecules of the self-assembled monolayer are highly ordered.

The term “fusion protein” refers to a protein composed of two or more polypeptides that, although typically unjoined in their native state, are joined by their respective amino and carboxyl termini through a peptide linkage to form a single continuous polypeptide. It is understood that the two or more polypeptide components can either be directly joined or indirectly joined through a peptide linker/spacer.

“Proteomics” means the study of or the characterization of either the proteome or some fraction of the proteome. The “proteome” is the total collection of the intracellular proteins of a cell or population of cells and the proteins secreted by the cell or population of cells. This characterization most typically includes measurements of the presence, and usually quantity, of the proteins which have been expressed by a cell. The function, structural characteristics (such as post translational modification), and location within the cell of the proteins can also be studied. “Functional proteomics” refers to the study of the functional characteristics, activity level, and structural characteristics of the protein expression products of a cell or population of cells.

The practice of this invention can involve the construction of recombinant nucleic acids and the expression of genes in transfected host cells. Molecular cloning techniques to achieve these ends are known in the art. A wide variety of cloning and in vitro amplification methods suitable for the construction of recombinant nucleic acids such as expression vectors are well-known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (2000 Supplement) (Ausubel).

### DESCRIPTION OF THE PREFERRED EMBODIMENTS

The invention provides for methods of immobilizing a polypeptide to a surface, arrays of such polypeptides, and kits for immobilizing a polypeptide to a surface, etc. The immobilized polypeptides of the invention provide significant advantages over previously available immobilized polypeptides and the methods for forming them. Previously available methods for producing polypeptide arrays required either step-wise synthesis of the polypeptide while immobilized on the surface, or nonspecific cross-linking to the support of functional groups present on side chains of amino acids present in a particular polypeptide. Both methods have significant disadvantages. Step-wise synthesis on a surface (e.g., a chip) is limited by the efficiency and accuracy of the available synthetic methods of peptide synthesis. As a practical matter, peptide synthesis methods are limited to peptides of about 60 amino acids and less. Moreover, it can be difficult or impossible to obtain proper secondary and tertiary structure of a protein that is synthesized by step-wise peptide synthesis.

Cross-linking functional groups on a polypeptide to a reactive group on a surface, the other major methods for immobilizing polypeptides on a surface is often problematic. An example of such methods involves the formation of a disulfide cross-link between cysteine residues present in the polypeptide and an immobilized thiol-containing group. Because the amino acid with the corresponding functional group can be found at multiple locations within a polypeptide, and/or can be present near a site necessary for biological activity of the polypeptide, cross-linking at all such sites can interfere with or even eliminate the biological activity.

Unlike previously available methods for forming polypeptide arrays, the methods of the present invention permit a polypeptide to be attached to a surface using a single discrete attachment point on the polypeptide. While the previous methods generally result in a polypeptide being attached to the surface at several amino acid residues (e.g., each cysteine residue present in the protein), the methods of the invention allow one to attach a polypeptide to a surface at a discrete point (e.g., its carboxy terminus). Thus, one can obtain arrays in which each polypeptide is identically oriented. The ability to attach one or more polypeptides in a single orientation and with only one attachment point greatly increases the ability to screen potential therapeutic or other agents for ability to interact with the polypeptides in the array.

The methods of the invention involve functionalizing a polypeptide with an ester or thioester at the point of desired attachment (e.g., the carboxy terminus of the polypeptide), and reacting the ester or thioester with a molecule that has a first nucleophilic group at the 2 or 3 position relative to a second nucleophilic group. An example of a suitable molecule for this purpose is a 2-aminonucleophile, such as a 2-aminothiol. This nucleophilic molecule can be used to attach the polypeptide to a solid support. The ester or thioester and the first nucleophilic group of the compound undergo a transesterification reaction, thus producing an intermediate in which the polypeptide is linked to the compound by an ester or thioester bond. The intermediate then undergoes a spontaneous rearrangement to form a more stable bond between the polypeptide and the second nucleophilic group on the compound. In other embodiments, the ester or thioester containing polypeptide is immobilized by contacting the polypeptide with an anchor molecule containing a reactive group such as a hydrazine group, a hydroxylamine, or an aminooxy group, etc.

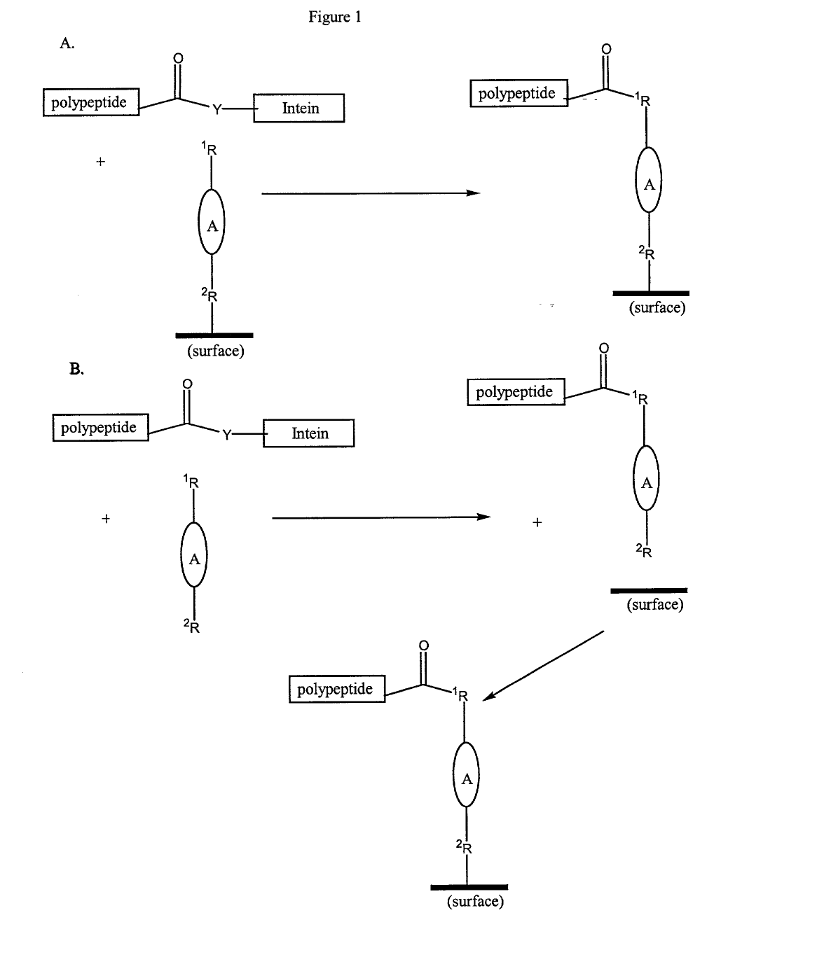

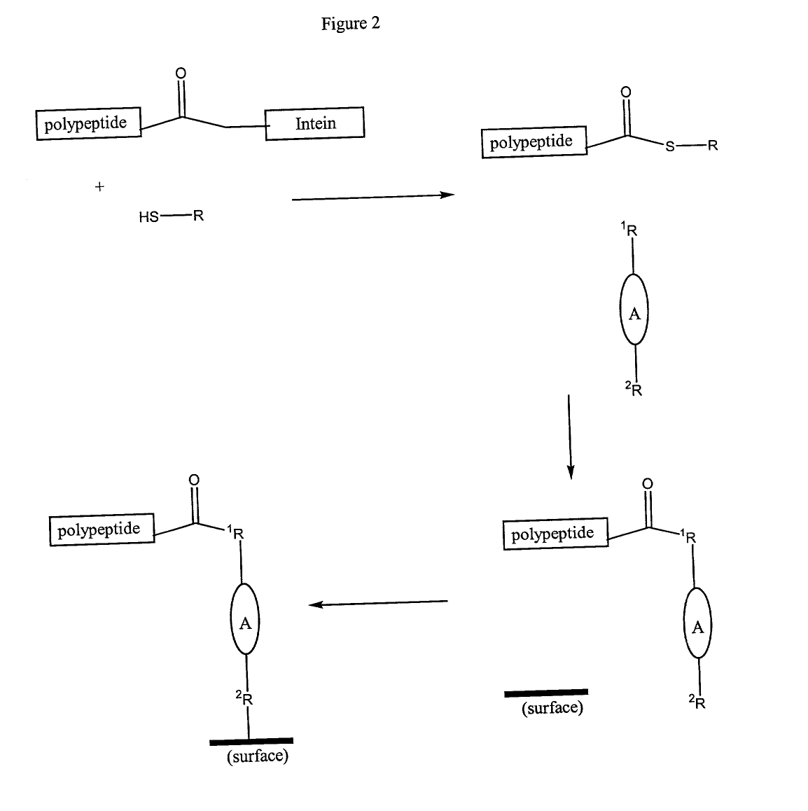

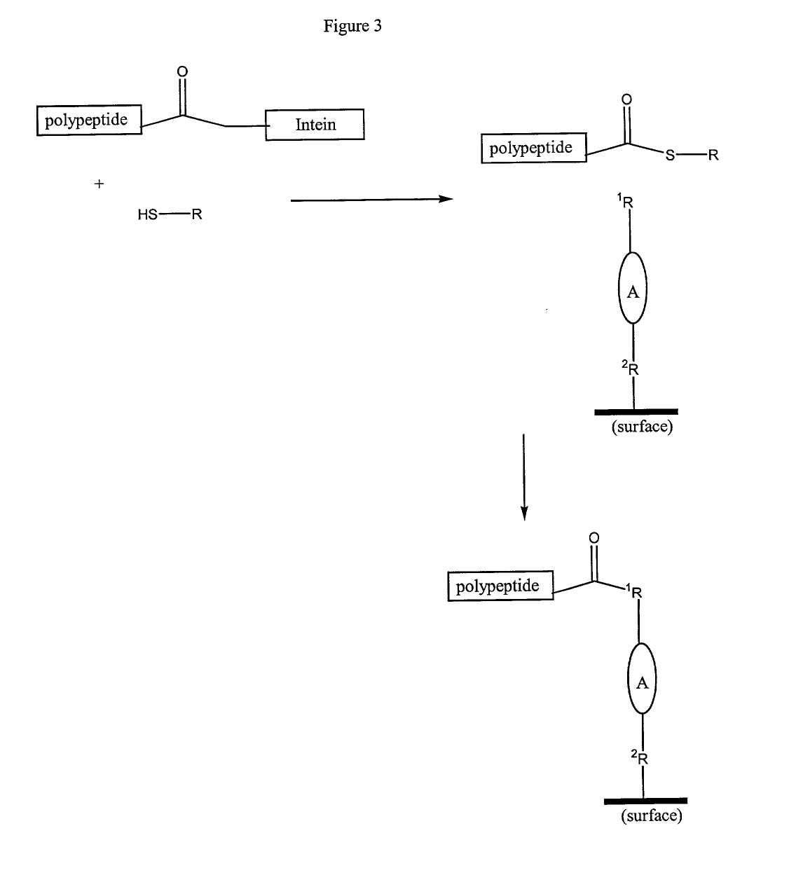

In certain embodiments, the thioester- or ester-containing polypeptide to be immobilized also comprises an intein, an intein fragment, or a mutated intein, etc (see e.g., FIG. 1). These intein-containing polypeptide are then reacted with a reactive group on the anchor molecule that is pre-immobilized to a surface or is subsequently immobilized to a surface (see, e.g., FIG. 1). In other embodiments, an activating compound is contacted with the polypeptide comprising an intein, an intein fragment, or a mutated intein (see FIGS. 2 and 3) prior to contact with the anchor molecule comprising a reactive group. The intein chemistry, anchor molecules, activating compounds, and reactive groups will be described in more detail below.

A. Derivatization of Polypeptides

The polypeptide arrays of the invention are made by introducing an ester group into the polypeptide at a specific position, generally at the carboxyl terminus of the polypeptide, and using this group to attach the polypeptide to a support. The ester group, as the term is used herein, can be any type of ester, including thioesters and the like, in addition to alcohol-derived esters.

1. Chemical Derivatization

The derivatization to introduce the ester or thioester group into the polypeptide can be accomplished in any of several ways. For example, chemical synthesis methods can be used to make a suitably derivatized polypeptide. Such methods are generally useful for relatively short polypeptides. One suitable method involves step-wise synthesis of a peptide on a resin that has an unoxidized thiol. The thiol is reacted with a protected amino acid succinimide to produce an aminothioester resin. The peptide is then synthesized on the resin, after which it is released with an appropriate compound to produce the desired peptide with a C-terminal thioester (see, e.g., WO 96/34878).

Chemical ligation provides another means by which a synthetic fragment (e.g., which contains an ester or thioester) can be joined to a polypeptide of interest (Dawson et al. (1994) Science 266: 776-779; Tam et al. (1995) Proc. Nat'l. Acad. Sci. USA 92: 12485-12489; Canne et al. (1996) J. Am. Chem. Soc. 118: 5891-5896; and Wilken and Hart (1998) Curr. Op. Biotechnol. 9: 412-426). For example, native chemical ligation involves the chemical ligation of an unoxidized N-terminal cysteine on a first polypeptide to a C-terminal thioester of a polypeptide of interest. A P-thioester intermediate is formed in which the first polypeptide is linked to the C-tenninus of the polypeptide. This intermediate undergoes a spontaneous intramolecular rearrangement, which results in the two molecules becoming linked by an amide bond (see, e.g., WO 96/34878). A catalytic thiol can be included in the reaction mixture. Native chemical ligation can be used, for example, to link a polypeptide that is derivatized to facilitate attachment to a solid support to a polypeptide of interest for analysis. The native chemical ligation reaction can be conducted before attaching the attachment polypeptide to a surface, or after attachment has occurred.

2. Intein-mediated Derivatization

In some embodiments, the polypeptide having an ester is obtained using inteins, which are also known as “protein introns,” “intervening protein sequences,” “protein spacers,” and the like. Inteins are somewhat analogous to introns found in mRNA molecules. As is the case for introns, inteins are spliced out of the respective polypeptide, resulting in joining of the portion of the polypeptide N-terminal to the intein (the “N-extein”) with the polypeptide portion that is to the C-terminal side of the intein (the “C-extein”). The splicing reaction involves an acyl rearrangement between the S or O side chain of a cysteine, threonine or serine residue at the N-terminal of the intein with the peptide bond which connects the Cys, Thr or Ser residue to the N-extein.

This rearrangement results in an intermediate in which the N-cysteine (or Ser or Thr) is attached to the adjacent extein by a thioester or ester, respectively. This intermediate then undergoes a trans-esterification reaction due to nucleophilic attack by an O or S-containing side chain of a Cys, Ser or Thr residue at the C-terminal end of the intein. This forms a branched polypeptide intermediate in which the N-extein is joined to a side chain of the Cys, Thr or Ser of the C-extein by a thioester or ester linkage. The intein is then released by cyclization of a conserved Asn residue at the carboxy end of the intein to form a succinimide derivative, followed by an O—N or S—N acyl shift and concomitant hydrolysis of the succinimide. The mechanisms of intein cleavage are discussed in, for example, Chong et al. (1998) Gene 192: 271-281; Evans et al. (1998) Protein Sci. 7: 2256-2264; and Paulus (1998) Chem. Soc. Reviews 27: 375-386.

Inteins are described in, for example, U.S. Pat. Nos. 5,981,182, and 5,834,247, which are herein incorporate by reference in their entirety for all purposes and for the purpose of teaching inteins and intein chemistry. Inteins generally include amino acid residues that are conserved among inteins of different proteins. Intein motifs are described in, for example, Pietrokovski, S. (1994) Protein Science 3:2340-2350; Perler et al. (1997) Nuc. Acids Res. 25:1087-93; Pietrokovski, S. (1998) Protein Sci. 7:64-71. Other methods of identifying inteins are described in, for example, Dalgaard et al. (1997) J. Computational Biol. 4:193-214 and Gorbalenya, A. E. (1998) Nucleic Acids Res 26:1741-8. “INBASE” a compilation of known inteins by New England Biolabs, is found at http://circuit.neb.com/inteins/int_id.html.

For use in the methods of the present invention, it is preferred to use mutant inteins in which only the amino-terminal end of the intein is capable of participating in the reaction. Such mutant inteins thus do not result in splicing of the N-extein to the C-extein. Instead, the N-extein is released from the intein upon attack by an activating compound that contains a nucleophilic group (e.g., a thiol or hydroxyl) under conditions conducive to intein cleavage. The activating compound then becomes attached to the end of the extein that was adjacent to the intein by a thioester or ester bond (see, e.g., Muir et al. (1998) Proc. Nat'l. Acad. Sci. USA 95: 6705-6710; Severinov and Muir (1998) J. Biol. Chem. 273: 16205-16209; Evans et al. (1998) Protein Sci. 7: 2256-2264). Suitable activating compounds that have nucleophilic groups include, for example, dithiothreitol (DTT), 2-mercaptoethanol, thiophenol, 2-mercaptoethanesulfonic acid, and cysteine-containing molecules, and the like. In some embodiments, the compounds contain 2-aminonucleophiles such as 2-aminothiols or 2-amino alcohols. These 2-aminonucleophiles can be attached to anchor molecules, such as are described in more detail below, which are used for attachment of the polypeptide to a support.

For some applications, the invention uses split inteins, in which the intein is split among two different polypeptides. The two molecules then undergo trans-splicing to excise the intein portions (termed the “n-intein” and the “c-intein”) and join the two exteins. For use in the invention, the polypeptide of interest is attached to an Int-n of a split intein and a molecule to be joined to the polypeptide (e.g., an anchor molecule) is attached to an Int-c of a split intein. The Int-n and the Int-c undergo the trans-splicing reaction, thus attaching the anchor molecule to the polypeptide. An example of a naturally occurring intein occurs in the DnaE polypeptide of Synechocystis, as described in Wu et al. (1998) Proc. Nat'l. Acad. Sci. USA 95: 9226-9231 and Gorbalenya (1998) Nucl. Acids Res. 26: 1741-1748. Other trans-spliced inteins also occur naturally and are likewise suitable for use in the invention. An intein that, in its natural form, is encoded as a single polypeptide with the associated exteins can also be split among two expression cassettes and used as a split intein (see, e.g., Gimble (1998) Chemistry and Biology 5: R251-R256).

The autoprocessing domains of hedgehog proteins are also useful for obtaining polypeptides that have an ester or thioester at its carboxyl terminus. These autoprocessing domains are similar to inteins, both in their structure and in their amino acid sequences. See, Porter et al. (1996) Cell 86: 21-34; Duan et al. (1997) Cell 89: 555-564; Hall et al. (1997) Cell 91: 85-97.

The use of split inteins in the methods of the present invention is particularly advantageous for attaching polypeptides that have disulfide bonds. Other attachment methods, e.g., attachment to sulfide groups and the like, often result in disruption of the naturally occurring disulfide bonds that occur in the polypeptide. Through use of a split intein, the joining of the anchor molecule is accomplished by intein-catalyzed splicing.

Generally, fusion proteins in which a polypeptide of interest is attached to a mutant intein are obtained by recombinant methods. A chimeric nucleic acid is constructed in which a polynucleotide that codes for the polypeptide of interest is upstream of, and in frame with, a coding region for an intein. Because intein-mediated cleavage is somewhat dependent upon the amino acid present at the end of the polypeptide of interest, the chimeric nucleic acid also can include one or more codons that add one or more amino acids which facilitate intein-mediated cleavage to the end of the target polypeptide. Examples of suitable amino acids for cleavage are described in, for example, New England Biolabs catalog entitled “IMPACT™-CN” (Beverly, Mass.). The chimeric nucleic acid is then expressed, resulting in biosynthesis of the fusion protein. The fusion protein is subjected to the cleavage reactions discussed herein to release the polypeptide of interest having an ester or thioester attached to the C-terminus. The polypeptide can then be attached to a surface as described herein.

The construction of suitable chimeric nucleic acids is facilitated by the use of an expression cassette. An “expression cassette” is a nucleic acid construct, generated recombinantly or synthetically, that has nucleic acid elements that are capable of effecting expression of a structural gene in host cells or other systems compatible with such sequences. Expression cassettes include at least promoters and optionally, transcription termination signals. Typically, a recombinant expression cassette includes a nucleic acid to be transcribed (e.g., a nucleic acid encoding a desired polypeptide), and a promoter. Additional factors necessary or helpful in effecting expression can also be used. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

In some embodiments, the expression cassette can also include a coding region for a tag that can noncovalently associate with a binding partner. Such tags are useful in the purification of the resulting polypeptide by affinity binding prior to immobilization on the array. Tags can also be used to attach the polypeptides to the surface to form the arrays, as discussed in more detail below. The tag coding region is typically present downstream of, and in frame with, the intein coding region. Upon expression, the protein can then be affinity purified using the tag, after which the intein-mediated cleavage releases the tag from the polypeptide to be immobilized.

Examples of suitable tags which are proteins include the binding domains of glutathione-S-transferase (GST), maltose-binding protein, chitinase (e.g., a chitin binding domain), cellulase (cellulose binding domain), thioredoxin, and the like. If the protein of interest an antibody or antibody fragment comprising an Fc region, then the tag may optionally be protein G, protein A, or recombinant protein AIG (a gene fusion product secreted from a non-pathogenic form of Bacillus which contains four Fc binding domains from protein A and two from protein G). Other examples of suitable fusion tags include T7 tag, S tag, His tag, PKA tag, HA tag, c-Myc tag, Trx tag, Hsv tag, Dsb tag, pelB/ompT, KSI, VSV-G tag, and β-Gal tag. A fusion protein that includes green fluorescent protein (GFP) or other proteins that can be visualized or can participate in a reaction which forms a detectable compound can be used for quantification of surface binding.

Examples of tag/tag binder pairs include, but are not limited to, the following:

| | |
|---|---|
| Fusion tags | Tag binders |
| Histidine(6-8 His) | NTA (Nitrilotriacetic acid, with a metal |
| | such as Ni, Co, Fe, Cu) |
| GST (220 aa) | GSH (Glutathione, 3 amino acids) |
| S-peptide (15 amino acids) | S (104 aa) |
| PKA peptide (5 amino acids; Protein Kinase | PKA |
| Inhibitor (PKI) peptide) | |
| HA peptide (9 amino acids) | HA |
| OligoPhenylalanine, or OligoLeucine (10-30 | KSI (125 aa) |
| amino acids) | |
| Arg (6-10 Arg) | OligoGlutamic acid (10-15 amino acids) |
| Asp (6-10 Asp) | OligoArginine (10-15 amino acids) |
| MBP (360 aa) | Maltose |
| GBD | Galactose |
| CBD (107-156 aa) | Cellulose |

Methods for constructing and expressing genes that encode fusion proteins are well known to those of skill in the art. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (2000 Supplement) (Ausubel); Cashion et al., U.S. Pat. No. 5,017,478; and Carr, European Patent No. 0,246,864.

The use of inteins is particularly suitable for constructing arrays of different protein species, such as those obtained through use of DNA shuffling, recombination, and other methods known to those of skill in the art for obtaining libraries of nucleic acids that encode different polypeptide species. The resulting libraries of polypeptide-encoding polynucleotides are introduced into an expression cassette which includes an insertion site (preferably one or more restriction enzyme cleavage sites) at which a member of the library of polynucleotides is introduced into the expression cassette. The insertion site is situated such that when a polynucleotide is introduced, at least in some fraction of cases, an open reading frame is formed in which the polypeptide-encoding open reading frame and that of the intein coding region are in the same frame. A library of cDNA molecules, genomic DNA fragment, polynucleotides that have been subjected to recombination, and the like, is ligated into the expression cassette and the resulting fusion protein expressed, subjected to intein-mediated cleavage to obtain the derivatized polypeptides, and immobilized on the surface for screening.

Chimeric nucleic acids that encode the polypeptide-intein fusion proteins can be expressed using either in vivo or in vitro expression systems. Many suitable expression vectors for expression of polypeptides such as the intein-containing fusion proteins are commercially available (from Qiagen, Novagen, Clontech, and many other companies). Suitable expression vectors and systems specifically designed for expression of intein-containing fusion proteins are commercially available from, for example, New England Biolabs (Beverly, Mass.). For in vivo expression, the vectors are introduced into cells of an appropriate organism which recognizes the expression control signals present in the expression cassette. Expression in vivo can be done in bacteria (for example, Escherichia coli, Bacillus sp., and the like), plants (for example, Nicotiana tabacum), lower eukaryotes (for example, Saccharomyces cerevisiae, Saccharomyces pombe, Pichia pastoris, and filamentous fungi), or higher eukaryotes (for example, baculovirus-infected insect cells, insect cells, mammalian cells). The choice of organism for optimal expression can depend on the extent of post-translational modifications (i.e., glycosylation, lipid-modifications) desired. One of ordinary skill in the art will be able to readily choose which host cell type is most suitable for the protein to be immobilized and application desired.

In other embodiments, in vitro expression systems are used. Systems have long been available for translation of mRNA molecules. Both eukaryotic and prokaryotic cell-free systems are available. Eukaryotic systems include, for example, the rabbit reticulocyte system (Pelham and Jackson (1976) Eur. J. Biochem., 67: 247-256) and the wheat germ lysate (Roberts and Paterson (1973) Proc. Nat'l. Acad. Sci. USA 70: 2330-2334). Prokaryotic systems include the E. coli S30 extract method and the fractionated method described by Gold and Schweiger (1971) Meth. Enzymol. 20: 537.

Coupled transcription and translation in vitro expression systems are particularly suitable for use in the present invention (see, e.g., U.S. Pat. No. 5,324,637; Kigawa and Yokohama (1991) J. Biochem. 110:166-168; Kudlicki et al. (1992) Anal Biochem. 206:389-393; and Pratt, J., “Coupled transcription-translation in prokaryotic cell-free systems” in Transcription & Translation: A Practical Approach, Hames & Higgins, IRL Press, Chapter 7, pp. 179-209 (1987). Suitable systems include, for example, Escherichia coli S30 lysates (see, e.g., Zubay (1973) Ann. Rev. Genet. 7: 267), such as, for example, those from strains that express the chimeric nucleic acid under the control of a T7 RNA polymerase promoter. Preferably, the strains are protease-deficient strain. Other systems include wheat germ lysates; reticulocyte lysates (see, e.g., Promega, Pharmacia, Panvera)).

In a presently preferred embodiment, the in vitro expression is conducted directly on a surface to which the polypeptide is to be immobilized. This can be accomplished, for example, using a nanodroplet technique that has been described for making a miniaturized array of cell-based assays (You et al. (1997) Chem. Biol. 4: 969-975). The methods of the invention can be performed by applying small droplets of a cell-free expression system to a surface. A micro tip can be used for the application of the droplets. If desired, the surface can be pre-coated with PDMS, polyethylene glycol, or other reagents known to reduce non-specific binding to a surface.

Avoidance of evaporation during the expression is of particular importance in the in vitro expression methods. To reduce evaporation, one can use microchannels to apply the cell free expression systems. Suitable microchannel dispensers, and surfaces for use with such dispensers, are described below and in U.S. patent application Ser. No. 09/792,335, filed Feb. 23, 2001. The cell-free systems can be pumped through microchannels to load a channel above the surface to which is attached the array of polypeptides. One can load different chambers with cell-free expression samples that contain different templates.

The invention also provides arrays in which a plurality of polypeptide species are attached to a surface, along with polynucleotides that encode each of the polypeptide species. Such arrays allow one to not only identify a polypeptide of interest by screening the array, but also identify the particular polynucleotide that encodes the polypeptide of interest. Thus, one can readily use the polynucleotide to determine the deduced amino acid sequence of the polypeptide, and to express the polypeptide in quantity.

The combined arrays can be made by conducting the in vitro expression directly on a surface to which the polypeptide is to be immobilized, as described above, while also attaching the polynucleotide to the surface. Methods for attaching polynucleotides to a surface are known to those of skill in the art.

3. Pre-screening of Polypeptides Prior to Attachment to Surface

It is sometimes desirable to conduct an initial screening of a polypeptide library to identify those that have a particular activity prior to immobilizing the polypeptide species in an array on a surface. Phage display and related methods are particularly amenable to such initial screening methods. A basic concept of display methods that use phage or other replicable genetic package is the establishment of a physical association between DNA encoding a polypeptide to be screened and the polypeptide. This physical association is provided by the replicable genetic package, which displays a polypeptide as part of a capsid enclosing the genome of the phage or other package, wherein the polypeptide is encoded by the genome. The establishment of a physical association between polypeptides and their genetic material allows simultaneous mass screening of very large numbers of phage bearing different polypeptides. Phage displaying a polypeptide with a desired activity, such as affinity to a target, e.g., a receptor, bind to the target and these phage are enriched by affinity screening to the target. The identity of polypeptides displayed from these phage can be determined from the respective phage genomes. Using these methods, a polypeptide identified as having a binding affinity for a desired target can then be synthesized in bulk by conventional means.

Typically, the initial screening using such methods involves expressing the recombinant peptides or polypeptides encoded by the recombinant polynucleotides of a library as fusions with a protein that is displayed on the surface of a replicable genetic package. For example, phage display can be used. See, e.g, Cwirla et al., Proc. Nat'l. Acad. Sci. USA 87: 6378-6382 (1990); Devlin et al., Science 249: 404-406 (1990), Scott & Smith, Science 249: 386-388 (1990); Ladner et al., U.S. Pat. No. 5,571,698. Other replicable genetic packages include, for example, bacteria, eukaryotic viruses, yeast, and spores.

The genetic packages most frequently used for display libraries are bacteriophage, particularly filamentous phage, and especially phage M13, Fd and Fl. Most work has involved inserting libraries encoding polypeptides to be displayed into either gIII or gVIII of these phage forming a fusion protein. See, e.g., Dower, WO 91/19818; Devlin, WO 91/18989; MacCafferty, WO 92/01047 (gene III); Huse, WO 92/06204; Kang, WO 92/18619 (gene VIII). Such a fusion protein comprises a signal sequence, usually but not necessarily, from the phage coat protein, a polypeptide to be displayed and either the gene III or gene VIII protein or a fragment thereof. Exogenous coding sequences are often inserted at or near the N-terminus of gene III or gene VIII although other insertion sites are possible.

Eukaryotic viruses can be used to display polypeptides in an analogous manner. For example, display of human heregulin fused to gp70 of Moloney murine leukemia virus has been reported by Han et al., Proc. Nat'l. Acad. Sci. USA 92: 9747-9751 (1995). Spores can also be used as replicable genetic packages. In this case, polypeptides are displayed from the outer surface of the spore. For example, spores from B. subtilis have been reported to be suitable. Sequences of coat proteins of these spores are described in Donovan et al., J. Mol. Biol. 196: 1-10 (1987). Cells can also be used as replicable genetic packages. Polypeptides to be displayed are inserted into a gene encoding a cell protein that is expressed on the cells surface. Bacterial cells including Salmonella typhimurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and especially Escherichia coli are preferred. Details of outer surface proteins are discussed by Ladner et al., U.S. Pat. No. 5,571,698 and references cited therein. For example, the lamB protein of E. coli is suitable.

Once the prescreening has identified polypeptides that are of interest for further screening, the polypeptides can be derivatized with a C-terminal ester or thioester and immobilized on a surface according to the methods of the invention. The polypeptides of interest can be released from the surface protein by methods known to those of skill in the art, such as proteolytic cleavage and the like. Chemical methods can then be used to accomplish the desired derivatization.

A more preferable way to obtain release of the polypeptide of interest while simultaneously accomplishing the introduction of a terminal ester or thioester is provided by the invention. An intein coding region is introduced between the polynucleotide of interest and the coding region for the surface-displayed protein. The resulted fusion protein, when expressed, then includes the polypeptide of interest (e.g., a library member, and the like), the intein, and the phage surface-displayed protein. After expression, the initial screening is conducted using the polypeptide displayed on the phage or other replicable genetic package. After identifying those phage that display a polypeptide that has the desired activity, the polypeptide is released from the phage simply by carrying out the intein cleavage reactions described herein. No proteolytic cleavage or other undesirable method is required. Moreover, the protein then has the desired ester or thioester bond which can serve as an attachment point.

The invention provides expression cassettes and expression vectors that facilitate the use of display on replicable genetic packages for initial screening, followed by intein-mediated derivatization of the polypeptide. The expression cassettes include an insertion site at which a member of the library of nucleic acids is introduced into the expression cassette. The insertion site preferably includes one or more restriction enzyme cleavage sites. Downstream of the insertion site is an intein coding region, which in turn is followed by an open reading frame that encodes a polypeptide that is displayed on a surface of a replicable genetic package. The introduction of coding region for a polypeptide of interest, such as a member of the library of nucleic acids, at the insertion site results in an open reading frame that encodes a fusion protein that comprises the polypeptide encoded by the library member, the intein, and the surface-displayed polypeptide.

The fusion protein is then expressed in the appropriate system which results in the polypeptide of interest being displayed on the surface of the corresponding replicable genetic package. After initial screening using methods known to those of skill in the art, the fusion proteins that are of interest for further evaluation and/or use are subjected to intein-mediated cleavage and ester/thioester derivatization, followed by attachment to a surface.

The target protein/intein/surface display peptide fusion proteins are useful not only for preselecting polypeptides for subsequent immobilization, but are also useful for modifying a protein by adding the phage display-selected polypeptide to an end of a protein of interest. After selection of individual phage that display polypeptides having the desired biological activity (e.g., binding activity), the polypeptides can be subjected to intein-mediated cleavage to release the binding polypeptides and simultaneously introduce a reactive ester or thioester group. The binding polypeptides can then be attached to a protein of interest.

B. Anchor Molecules and Attachment to Surface

The ester- or thioester-containing polypeptides are attached to a surface by reacting the ester or thioester groups with an anchor molecule comprising a reactive group (e.g., a functional group) that reacts with the ester or thioester group to attach the polypeptide to the anchor molecule. The anchor molecule can be attached to the surface before, after, or during reaction with the ester or thioester.

In certain embodiments, the reactive group on the anchor molecule is a group that has a nucleophilic group at the 2 or 3 position relative to a second nucleophilic group. One of the nucleophilic groups is, in some embodiments, a to a carbonyl group. One nucleophilic group on the compound attacks the ester or thioester on the polypeptide to form an intermediate, which then undergoes an intramolecular rearrangement involving the second nucleophile on the compound. The intermediate typically involves a 5- or 6-membered ring structure. The first reaction involves the group that has the greatest nucleophilic character, while the second nucleophilic group generally forms a more thermodynamically and/or kinetically stable product than the first. For example, a 2-aminonucleophile or 3-aminonucleophile compound (e.g., 2-aminothiol or 3-aminothiol) can undergo a trans-esterification reaction with the ester or thioester on the polypeptide. This reaction produces an intermediate in which the polypeptide is linked to the compound by a 2-aminonucleophile-ester bond. The resulting 2-aminonucleophile-ester bond then undergoes an intramolecular rearrangement mediated by the second nucleophilic group on the compound to form an amide bond that stably links the anchor molecule to the polypeptide. For illustrative purposes, examples of suitable compounds that have two nucleophilic groups include structures such as:

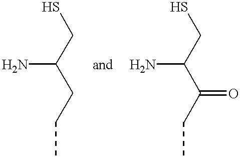

The above structures can also have additional substitutions at one or more of the carbons, and can have an additional carbon between the amine and the thiol. Examples of suitable nucleophilic groups include those known to those of skill in the art, including O, S, N, and Se, for example. The dashed lines represent a moiety that is, or can be, attached to a surface.

In other embodiments, the reactive group on the anchor molecule is a nucleophilic group that can directly react with the thioester or ester. Examples of such reactive groups, include without limitation, hydrazine groups (e.g., NH₂NH—R, where R is the anchor molecule), hydroxylamine groups, and aminooxy groups, etc.

The anchor molecules having two nucleophilic reactive groups or containing reactive groups such as a hydrazine, a hydroxlamine, or an aminooxy group, etc. can be either directly attachable to a surface, or can be attached to a surface by another compound with which the di-nucleophilic compound can react. For example, the di-nucleophilic compound can be covalently linked to the surface-attached compound, or can be noncovalently associated to the surface-attached compound. For example, the di-nucleophilic compound can include a functional group that can form a covalent bond with a molecule attached to a surface. Preferably, the functional group is one that can participate in a chemoselective ligation reaction having little or no cross reactivity with functional groups present in the amino acids that make up the polypeptide being attached. Alternatively, the reactive functional groups can exert some cross reactivity if the groups are activated in proximity to the desired target under conditions wherein bond formation with the target is favored over reactivity with other sites. Examples of such reactive groups (or covalent linking groups) include ketones (which can react with an acyl hydrazine on a surface to form an acyl hydrazone), olefins (which can react with a second olefin on a surface or as part of a label in a cross olefin metathesis catalyzed by, for example, a ruthenium complex), or a diketone (which can react with a guanidine group). Of course, one can reverse which member of the reactive pairs is attached to the surface, and attach an acyl hydrazine, for example, to the di-nucleophilic compound and the ketone to the surface. Other covalent linking groups useful in the present invention include epoxides, aldehydes, reactive esters (e.g., pentafluorophenyl esters, nitrophenyl esters), isocyanates and thioisocyanates, carboxylic acid chlorides, dissulfides and sulfonate esters (e.g, mesylates, tosylates and the like). Still other covalent linking groups are the sulfhydryl groups (preferably protected until reaction is desired). Other suitable covalent linking groups include, but are not limited to, maleimide, isomaleimide, N-hydroxysuccinimide (Wagner et al. (1996) Biophysical Journal 70: 2052-2066), nitrilotriacetic acid (U.S. Pat. No. 5,620,850), activated hydroxyl, haloacetyl, activated carboxyl, hydrazide, epoxy, aziridine, sulfonylchloride, trifluoromethyldiaziridine, pyridyldisulfide, N-acyl-imidazole, imidazolecarbamate, vinylsulfone, succinimidylcarbonate, arylazide, anhydride, diazoacetate, benzophenone, isothiocyanate, isocyanate, imidoester, fluorobenzene, and the like.

The functional group will in some embodiments be protected, or otherwise rendered inactive to covalent bond formation, by a protecting group. A variety of protecting groups are useful in the invention and can be selected based on the functionality present in the functional group. The term “protecting group” as used herein, refers to any of the groups which are designed to block one reactive site in a molecule while a chemical reaction is carried out at another reactive site. More particularly, the protecting groups used herein can be any of those groups described in Greene et al., Protective Groups In Organic Chemistry, 2nd Ed., John Wiley & Sons, New York, N.Y., 1991. The proper selection of protecting groups for a particular synthesis will be governed by the overall methods employed in the synthesis. For example, in automated synthesis photolabile protecting groups such as NVOC, MeNPOC, and the like can be used. In other embodiments, protecting groups may used that are removable by chemical methods, such as FMOC, DMT and other methods known to those of skill in the art.

In some embodiments, the di-nucleophilic compound is a peptide that has at its amino terminus a Cys, Ser, or Thr residue which can undergo the trans-esterification reaction with the polypeptide to be immobilized. The peptide can have attached, generally at its carboxyl terminus, a functional group such as those described above which can form a covalent linkage with a molecule that is attached to a surface. Alternatively, the peptide can include a tag which can non-covalently associate with a molecule that is attached to a surface. Suitable tags and respective binding partners are known to those of skill in the art, and several examples are described above.

The polypeptides to be immobilized can be attached to the di-nucleophilic compounds prior to, simultaneously with, or after the di-nucleophilic compounds are attached to the surface.

Methods of attaching molecules to different surfaces are known to those of skill in the art. In some embodiments, an organic thinfilm is employed to forms a layer either on the substrate itself or on a coating covering the substrate, upon which each of the patches of polypeptides is immobilized. Organic thinfilms are described in copending U.S. patent application Ser. No. 09/820,210, filed Mar. 27, 2001. A variety of different organic thinfilms are suitable for use in the present invention. Methods for the formation of organic thinfilms include in situ growth from the surface, deposition by physisorption, spin-coating, chemisorption, self-assembly, or plasma-initiated polymerization from gas phase. For instance, a hydrogel composed of a material such as dextran can serve as a suitable organic thinfilm on the patches of the array. In one preferred embodiment of the invention, the organic thinfilm is a lipid bilayer. In another preferred embodiment, the organic thinfilm of each of the patches of the array is a monolayer. A monolayer of polyarginine or polylysine adsorbed on a negatively charged substrate or coating is one option for the organic thinfilm. Another option is a disordered monolayer of tethered polymer chains. In a particularly preferred embodiment, the organic thinfilm is a self-assembled monolayer. A monolayer of polylysine is one option for the organic thinfilm. The organic thinfilm can be, for example, a self-assembled monolayer which comprises molecules of the formula X-R-Y, wherein R is a spacer, X is a functional group that binds R to the surface, and Y is a molecule that attaches to the polypeptide, or a moiety attached to the polypeptide. For example, Y can be the dinucleophilic compound which is used to attach the polypeptides onto the monolayer, or Y can be a binding partner for a tag that is attached to the polypeptide.

In an alternative embodiment, the self-assembled monolayer is comprised of molecules of the formula (X)ₐR(Y)_{b} where a and b are, independently, integers greater than or equal to 1 and X, R, and Y are as previously defined. In another alternative embodiment, the organic thinfilm comprises a combination of organic thinfilms such as a combination of a lipid bilayer immobilized on top of a self-assembled monolayer of molecules of the formula X-R-Y. As another example, a monolayer of polylysine can also optionally be combined with a self-assembled monolayer of molecules of the formula X-R-Y (see U.S. Pat. No. 5,629,213).

In all cases, the coating, or the substrate itself if no coating is present, must be compatible with the chemical or physical adsorption of the organic thinfilm on its surface. For instance, if the patches comprise a coating between the substrate and a monolayer of molecules of the formula X-R-Y, then it is understood that the coating must be composed of a material for which a suitable functional group X is available. If no such coating is present, then it is understood that the substrate must be composed of a material for which a suitable functional group X is available.

The methods of the invention can also be used with trifunctional linkers such as are described in copending U.S. patent application Ser. No. 09/820,210, filed Mar. 27, 2001. These linkers are useful for the site-specific introduction of a label to a polypeptide, in addition to the site-specific immobilization of a polypeptide to a solid support. These trifunctional crosslinking groups have, in some embodiments, the formula:

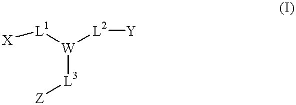

wherein W is a trivalent core component; L¹, L² and L³ are independently linking groups; X is a non-covalent polypeptide tag binder; Y is a photoactivatable covalent linking group; and Z is a protected or unprotected covalent crosslinking group. In this particular example, a trifunctional linking group is depicted having three functional groups (X, Y and Z) attached via linkers (L¹, L² and L³) to a central core (W). The first functional group is one which provides a non-covalent association with a targeted polypeptide or a polypeptide of interest. For example, the trifunctional linking group can form a non-covalent association complex with a polypeptide having a suitable tag (e.g., a his-tag). The second functional group can then establish a covalent linkage to the polypeptide at a site which is proximate to the initial non-covalent association site. One of skill in the art will appreciate that although the polypeptide is shown as a relatively small circle (relative to the size of the trifunctional crosslinking group), in fact the polypeptide in most embodiments is quite large relative to the crosslinking group. Nevertheless, the site for covalent attachment of functional group Y will depend on the lengths and flexibility of the linking groups L¹ and L². Typically, the site for covalent attachment of Y to the polypeptide will be within about 50 Å of the site of non-covalent association. Release of the non-covalent functional group (X) from the polypeptide provides a polypeptide having a covalently bound trifunctional crosslinking group. In subsequent steps, functional group Z of the polypeptide-crosslinking group composition can be used, for example, to attach a suitable label to the polypeptide, or to immobilize the polypeptide on a suitable support.

C Polypeptide Arrays

The present invention provides arrays of polypeptides, as well as methods for synthesizing such arrays. Typically, the polypeptide arrays comprise micrometer-scale, two-dimensional patterns of patches of polypeptides immobilized on a surface of the substrate. Polypeptide arrays and their use for high-throughput screening are described in, for example, co-pending U.S. patent application Ser. No. 09/115,455, filed Jul. 14, 1998; Ser. No. 09/353,215, filed Jul. 14, 1999 and Ser. No. 09/353,555, filed Jul. 14, 1999; and related PCT published applications WO 00/04382, WO 00/04389 and WO 00/04390).

In one embodiment, the present invention provides an array of polypeptides which comprises a substrate, at least one organic thinfilm on some or all of the substrate surface, and a plurality of patches arranged in discrete, known regions on portions of the substrate surface covered by organic thinfilm, wherein each of said patches comprises a polypeptide immobilized on the underlying organic thinfilm.

In most cases, the array will comprise at least about ten patches. In a preferred embodiment, the array comprises at least about 50 patches. In a particularly preferred embodiment the array comprises at least about 100 patches. In alternative preferred embodiments, the array of polypeptides can comprise more than 10³, 10⁴ or 10⁵ patches.

The area of surface of the substrate covered by each of the patches is preferably no more than about 0.25 mm². Preferably, the area of the substrate surface covered by each of the patches is between about 1 μm² and about 10,000 μm². In a particularly preferred embodiment, each patch covers an area of the substrate surface from about 100 μm² to about 2,500 μm². In an alternative embodiment, a patch on the array can cover an area of the substrate surface as small as about 2,500 nm², although patches of such small size are generally not necessary for the use of the array.

The patches of the array can be of any geometric shape. For instance, the patches can be rectangular or circular. The patches of the array can also be irregularly shaped.

The distance separating the patches of the array can vary. Preferably, the patches of the array are separated from neighboring patches by about 1 μm to about 500 μm. Typically, the distance separating the patches is roughly proportional to the diameter or side length of the patches on the array if the patches have dimensions greater than about 10 μm. If the patch size is smaller, then the distance separating the patches will typically be larger than the dimensions of the patch.

In a preferred embodiment of the array, the patches of the array are all contained within an area of about 1 cm² or less on the surface of the substrate. In one preferred embodiment of the array, therefore, the array comprises 100 or more patches within a total area of about 1 cm² or less on the surface of the substrate. Alternatively, a particularly preferred array comprises 10³ or more patches within a total area of about 1 cm² or less. A preferred array can even optionally comprise 10⁴ or 10⁵ or more patches within an area of about 1 cm²r less on the surface of the substrate. In other embodiments of the invention, all of the patches of the array are contained within an area of about 1 m² or less on the surface of the substrate.

Typically, only one type of polypeptide is immobilized on each patch of the array. In a preferred embodiment of the array, the polypeptide immobilized on one patch differs from the polypeptide immobilized on a second patch of the same array. In such an embodiment, a plurality of different polypeptides are present on separate patches of the array. Typically the array comprises at least about ten different polypeptides. Preferably, the array comprises at least about 50 different polypeptides. More preferably, the array comprises at least about 100 different polypeptides. Alternative preferred arrays comprise more than about 10³ different polypeptides or more than about 10⁴ different polypeptides. The array can even optionally comprise more than about 10⁵ different polypeptides.

In one embodiment of the array, each of the patches of the array comprises a different polypeptide. For instance, an array comprising about 100 patches could comprise about 100 different polypeptides. Likewise, an array of about 10,000 patches could comprise about 10,000 different polypeptides. In an alternative embodiment, however, each different polypeptide is immobilized on more than one separate patch on the array. For instance, each different polypeptide can optionally be present on two to six different patches. An array of the invention, therefore, can comprise about three-thousand polypeptide patches, but only comprise about one thousand different polypeptides since each different polypeptide is present on three different patches.

In another embodiment of the present invention, although the polypeptide of one patch is different from that of another, the polypeptides are related. In a preferred embodiment, the two different polypeptides are members of the same polypeptide family. The different polypeptides on the invention array can be either functionally related or just suspected of being functionally related. In another embodiment of the invention array, however, the function of the immobilized polypeptides can be unknown. In this case, the different polypeptides on the different patches of the array share a similarity in structure or sequence or are simply suspected of sharing a similarity in structure or sequence. Alternatively, the immobilized polypeptides can be just fragments of different members of a polypeptide family.

The polypeptides immobilized on the array of the invention can be members of a polypeptide family such as a receptor family (examples: growth factor receptors, catecholamine receptors, amino acid derivative receptors, cytokine receptors, lectins), ligand family (examples: cytokines, serpins), enzyme family (examples: proteases, kinases, phosphatases, ras-like GTPases, hydrolases), and transcription factors (examples: steroid hormone receptors, heat-shock transcription factors, zinc-finger proteins, leucine-zipper proteins, homeodomain proteins). In one embodiment, the different immobilized polypeptides are all HIV proteases or hepatitis C virus (HCV) proteases. In other embodiments of the invention, the immobilized polypeptides on the patches of the array are all hormone receptors, neurotransmitter receptors, extracellular matrix receptors, antibodies, DNA-binding proteins, intracellular signal transduction modulators and effectors, apoptosis-related factors, DNA synthesis factors, DNA repair factors, DNA recombination factors, or cell-surface antigens.

In some embodiments, the polypeptide immobilized on each patch is an antibody or antibody fragment. The antibodies or antibody fragments of the array can optionally be single-chain Fvs, Fab fragments, Fab′ fragments, F(ab′)2 fragments, Fv fragments, dsFvs diabodies, Fc fragments, full-length, antigen-specific polyclonal antibodies, or full-length monoclonal antibodies. In a preferred embodiment, the immobilized polypeptides on the patches of the array are monoclonal antibodies, Fab fragments or single-chain Fvs.

In another preferred embodiment of the invention, the polypeptides immobilized to each patch of the array are polypeptide-capture agents.

In an alternative embodiment of the invention array, the polypeptides on different patches are identical.

Biosensors, micromachined devices, and diagnostic devices that comprise the polypeptide arrays of the invention are also contemplated by the present invention.

The physical structure of the polypeptide arrays will typically comprise a substrate and, optionally, a coating or organic thin film or both.

The substrate of the array can be either organic or inorganic, biological or non-biological, or any combination of these materials. In one embodiment, the substrate is transparent or translucent. The portion of the surface of the substrate on which the patches reside is preferably flat and firm or semi-firm. However, the array of the prevent invention need not necessarily be flat or entirely two-dimensional. Significant topological features can be present on the surface of the substrate surrounding the patches, between the patches or beneath the patches. For instance, walls or other barriers can separate the patches of the array.

Numerous materials are suitable for use as a substrate in the array embodiment of the invention. For instance, the substrate of the invention array can comprise a material selected from a group consisting of silicon, silica, quartz, glass, controlled pore glass, carbon, alumina, titania, tantalum oxide, germanium, silicon nitride, zeolites, and gallium arsenide. Many metals such as gold, platinum, aluminum, copper, titanium, and their alloys are also options for substrates of the array. In addition, many ceramics and polymers can also be used as substrates. Polymers which can be used as substrates include, but are not limited to, the following: polystyrene; poly(tetra)fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyatkenesulfone (PAS); polypropylene; polyethylene; polyhydroxyethylmethacrylate (HEMA); polydimethylsiloxane; polyacrylamide; polyimide; and block-copolymers. Preferred substrates for the array include silicon, silica, glass, and polymers. The substrate on which the patches reside can also be a combination of any of the aforementioned substrate materials.

An array of the present invention can optionally further comprise a coating between the substrate and organic thinfilm on the array. This coating can either be formed on the substrate or applied to the substrate. The substrate can be modified with a coating by using thin-film technology based, for example, on physical vapor deposition (PVD), thermal processing, or plasma-enhanced chemical vapor deposition (PECVD). Alternatively, plasma exposure can be used to directly activate or alter the substrate and create a coating. For instance, plasma etch procedures can be used to oxidize a polymeric surface (i.e., polystyrene or polyethylene to expose polar functionalities such as hydroxyls, carboxylic acids, aldehydes and the like).

The coating is optionally a metal film. Possible metal films include aluminum, chromium, titanium, tantalum, nickel, stainless steel, zinc, lead, iron, copper, magnesium, manganese, cadmium, tungsten, cobalt, and alloys or oxides thereof. In a preferred embodiment, the metal film is a noble metal film. Noble metals that can be used for a coating include, but are not limited to, gold, platinum, silver, and copper. In an especially preferred embodiment, the coating comprises gold or a gold alloy. Electron-beam evaporation can be used to provide a thin coating of gold on the surface of the substrate. In a preferred embodiment, the metal film is from about 50 nm to about 500 nm in thickness. In an alternative embodiment, the metal film is from about 1 nm to about 1 μm in thickness.

In alternative embodiments, the coating comprises a composition selected from the group consisting of silicon, silicon oxide, titania, tantalum oxide, silicon nitride, silicon hydride, indium tin oxide, magnesium oxide, alumina, glass, hydroxylated surfaces, and polymers.

In one embodiment of the invention array, the surface of the coating is atomically flat. In this embodiment, the mean roughness of the surface of the coating is less than about 5 angstroms for areas of at least 25 μm². In a preferred embodiment, the mean roughness of the surface of the coating is less than about 3 angstroms for areas of at least 25 μm². The ultraflat coating can optionally be a template-stripped surface as described in Heguer et al., Surface Science, 1993, 291:39-46 and Wagner et al., Langmuir, 1995, 11:3867-3875, both of which are incorporated herein by reference.

It is contemplated that the coatings of many arrays will require the addition of at least one adhesion layer between said coating and the substrate. Typically, the adhesion layer will be at least 6 angstroms thick and can be much thicker. For instance, a layer of titanium or chromium can be desirable between a silicon wafer and a gold coating. In an alternative embodiment, an epoxy glue such as Epo-tek 377®, Epo-tek 301-2®, (Epoxy Technology Inc., Billerica, Mass.) can be preferred to aid adherence of the coating to the substrate. Determinations as to what material should be used for the adhesion layer would be obvious to one skilled in the art once materials are chosen for both the substrate and coating. In other embodiments, additional adhesion mediators or interlayers can be necessary to improve the optical properties of the array, for instance, in waveguides for detection purposes.

Deposition or formation of the coating (if present) on the substrate is performed prior to the formation of the organic thinfilm thereon. Several different types of coating can be combined on the surface. The coating can cover the whole surface of the substrate or only parts of it. The pattern of the coating may or may not be identical to the pattern of organic thinfilms used to immobilize the polypeptides. In one embodiment of the invention, the coating covers the substrate surface only at the site of the patches of the immobilized. Techniques useful for the formation of coated patches on the surface of the substrate which are organic thinfilm compatible are well known to those of ordinary skill in the art. For instance, the patches of coatings on the substrate can optionally be fabricated by photolithography, micromolding (PCT Publication WO 96/29629), wet chemical or dry etching, or any combination of these.

The organic thinfilm on which each of the patches of polypeptides is immobilized forms a layer either on the substrate itself or on a coating covering the substrate. The organic thinfilm on which the polypeptides of the patches are immobilized is preferably less than about 20 nm thick. In some embodiments of the invention, the organic thinfilm of each of the patches can be less than about 10 nm thick.

A variety of different organic thinfilms are suitable for use in the present invention. Methods for the formation of organic thinfilms include in situ growth from the surface, deposition by physisorption, spin-coating, chemisorption, self-assembly, or plasma-initiated polymerization from gas phase. For instance, a hydrogel composed of a material such as dextran can serve as a suitable organic thinfilm on the patches of the array. In one preferred embodiment of the invention, the organic thinfilm is a lipid bilayer. In another preferred embodiment, the organic thinfilm of each of the patches of the array is a monolayer. A monolayer of polyarginine or polylysine adsorbed on a negatively charged substrate or coating is one option for the organic thinfilm. Another option is a disordered monolayer of tethered polymer chains. In a particularly preferred embodiment, the organic thinfilm is a self-assembled monolayer. A monolayer of polylysine is one option for the organic thinfilm.

In all cases, the coating, or the substrate itself if no coating is present, must be compatible with the chemical or physical adsorption of the organic thinfilm on its surface. For instance, if the patches comprise a coating between the substrate and a monolayer of molecules of the formula I, then it is understood that the coating must be composed of a material capable of binding the trifunctional crosslinking group of formula I. If no such coating is present, then it is understood that the substrate must be composed of a material which can covalently bind the trifunctional crosslinking group.

In a preferred embodiment of the invention, the regions of the substrate surface, or coating surface, which separate the patches of polypeptides are free of organic thinfilm. In an alternative embodiment, the organic thinfilm extends beyond the area of the substrate surface, or coating surface if present, covered by the polypeptide patches. For instance, optionally, the entire surface of the array can be covered by an organic thinfilm on which the plurality of spatially distinct patches of polypeptides reside. An organic thinfilm which covers the entire surface of the array can be homogenous or can optionally comprise patches of differing exposed functionalities useful in the immobilization of patches of different polypeptides. In still another alternative embodiment, the regions of the substrate surface, or coating surface if a coating is present, between the patches of polypeptides are covered by an organic thinfilm, but an organic thinfilm of a different type than that of the patches of polypeptides. For instance, the surfaces between the patches of polypeptides can be coated with an organic thinfilm characterized by low non-specific binding properties for polypeptides and other analytes.

A variety of techniques can be used to generate patches of organic thinfilm on the surface of the substrate or on the surface of a coating on the substrate. These techniques are well known to those skilled in the art and will vary depending upon the nature of the organic thinfilm, the substrate, and the coating if present. The techniques will also vary depending on the structure of the underlying substrate and the pattern of any coating present on the substrate. For instance, patches of a coating which is highly reactive with an organic thinfilm can have already been produced on the substrate surface. Arrays of patches of organic thinfilm can optionally be created by microfluidics printing, microstamping (U.S. Pat. Nos. 5,512,131 and 5,731,152), or microcontact printing (p.CP) (PCT Publication WO 96/29629). Subsequent immobilization of polypeptides to the reactive monolayer patches results in two-dimensional arrays of the agents. Inkjet printer heads provide another option for patterning monolayer molecules, or components thereof, or other organic thinfilm components to nanometer or micrometer scale sites on the surface of the substrate or coating (Lemmo et al., Anal Chem., 1997, 69:543-551; U.S. Pat. Nos. 5,843,767 and 5,837,860). In some cases, commercially available arrayers based on capillary dispensing (for instance, OmniGrid™ from Genemachines, inc, San Carlos, Calif., and High-Throughput Microarrayer from Intelligent Bio-Instruments, Cambridge, Mass.) can also be of use in directing components of organic thinfilms to spatially distinct regions of the array.

Diffusion boundaries between the patches of polypeptides immobilized on organic thinfilms such as self-assembled monolayers can be integrated as topographic patterns (physical barriers) or surface functionalities with orthogonal wetting behavior (chemical barriers). For instance, walls of substrate material or photoresist can be used to separate some of the patches from some of the others or all of the patches from each other. Alternatively, non-bioreactive organic thinfilms, such as monolayers, with different wettability can be used to separate patches from one another.

In some embodiments, the polypeptide species are attached to a chip that has a non-sample surface and a plurality of sample portions that are elevated with respect to the non-sample surface. Suitable chips, which are described in co-pending U.S. patent application Ser. No. 09/792,335, filed Feb. 23, 2001, generally include an array of reactive surfaces on the tops of pillars of well-defined dimensions. The tops of the pillars consist of, or are coated with, an interface layer capable of binding or adsorbing, or reacting with molecules contained in the material in channels that are present in a dispenser, as described therein. The pillar walls in the base between the pillars are designed either by structural topography, material choice, or surface coatings, in such a way that they minimize or prevent liquid cross-contamination between the individual pillars during the transfer or reaction step when the dispenser and chip are engaged. Using the same design techniques, these areas of the chip are also made resistant to the adsorption of the molecules or materials to be transferred or reacted. Together, these design features will prevent contamination between the top surfaces of the pillars. Thus, the biochips includes a topographical design wherein elevated surfaces or pillars are provided for isolating various materials and chemical reactions for observation and analysis.

Microfluid dispensers for providing materials in fluid form to the pillars are also described in U.S. patent application Ser. No. 09/792,335, filed Feb. 23, 2001. The dispensers can be used to create a final biochip with materials on the pillars for later analysis or chemical reactions, can be used to create the chemical reactions, and can further be used to observe and analyze the chemical reactions. By using the dispenser with a flow-cell adaptor that introduces analytes to the capture sites on top of the pillars, one can easily avoid non-specific binding of analytes on the sides of the pillars or the substrate between pillars.

D. Screening Methods

Arrays of surface-attached polypeptide species that are obtained using the methods of the invention are typically screened to identify those that have a desired activity (e.g., binding affinity to a target molecule of interest). Binding of a target molecule to the polypeptides of the arrays can be detected in a number of methods known to those of skill in the art. In one embodiment, fluorescent tags can be attached to known targets and binding can be measured by detecting fluorescence. Alternatively, ellipsometry (see, e.g., Elwing, H. Biomaterials 19(4-5):397-406 (1998); Werner, C. et al. Int. J Artif. Organs 22(3):160-176 (1999); and Ostroff, R. M. et al. Clin. Chem. 45(9):1659-64 (1999)) or surface plasmon resonance spectroscopy (see e.g., Mrksich, M.;, et al., Langmair 1995, 4383; Mrksich, M., et al., J. Am. Chem.Soc. 1995,117:12009; Sigal, G. B., et al., Anal. Chem. 1996, 68: 490) can also be used to detect binding events (e.g., on surfaces). These assays are particularly useful in detecting target molecules in complex mixtures such as blood or other bodily fluids.

The present invention also provides transferring the target molecule to a reaction chamber(s) that, in one embodiment, provides solutions or condition (e.g. elevated temperature) that dissociates the target molecule from the affinity molecule. The target molecule can then be detected using, e.g., liquid chromatography mass spectrometry (see, e.g., Niessen,. W. M. J. Chromatogr. A. 856(1-2):179-97 (1999) and Maurer H. H. J. Chromatogr. B. Biomed. Sci. Appli. 713(1):3-25 (1998)) or other methods known to those of skill in the art.

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

1. Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 12331250).

Preparation and screening of combinatorial chemical libraries are well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random biooligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 69096913), vinylogous polypeptides (Hagihara et aL (1992) J. Amer. Chem. Soc. 114: 6568), nonpeptidal peptidomimetics with a Beta D Glucose scaffolding (Hirschmann et al., (1992) J. Amer. Chem. Soc. 114: 92179218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries, peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g. Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

2. High Throughput Assays of Chemical Libraries

A variety of assays can be used to measure the interaction of different molecular components, e.g., to identify compounds that bind or inhibit gene products or that interact with a specific molecule. High throughput assays for the presence, absence, or quantification of particular nucleic acids or polypeptide products are well known to those of skill in the art. Similarly, binding assays are similarly well known. Thus, for example, U.S. Pat. No. 5,559,410 discloses high throughput screening methods for polypeptides, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

A discussion of the above technology and other relevant aspects of technology related to the present invention can be found in PCT Publication No. WO 200004382, entitled Arrays Of Proteins And Methods Of Use Thereof, Wagner, P. et al.; PCT Publication No. WO 200004389, entitled Arrays Of Protein-Capture Agents And Methods Of Use Thereof, Wagner, P. et al.; and PCT Publication No. WO 200004390 entitled Micro Devices For Screening Biomolecules, Wagner, P. et al.

E. Kits

The present invention further provides for kits to be supplied to end users for attaching polypeptides described herein to surfaces of substrates in a manner as provided by the methods herein disclosed. Kits may supply reagents including, for example, anchor molecule reagents, activating compounds and agents for activating polypeptide esters or thioesters, or for activating components, including surface attachment functional groups orthogonally from anchor molecule/polypeptide ligation groups, substrates, including substrates pre-derivatized with anchor molecules and/or substrates ready to receive anchor molecules, and instructions.

Other embodiments of kits include providing polypeptides containing an ester or thioester along components including instructions, anchor molecules, substrates, anchor molecule derivatized substrates, or where the polypeptide has been modified with the anchor molecule.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method for immobilizing a polypeptide to a surface, wherein the method comprises:
contacting a polypeptide which comprises an ester or thioester, with an anchor molecule comprising a first nucleophilic group at a 2 or 3 position relative to a second nucleophilic group,
wherein the ester or thioester undergoes a trans-esterification reaction with the first nucleophilic group, thus forming an intermediate compound in which the polypeptide is attached to the anchor molecule through the first nucleophilic group; and
attaching the anchor molecule to a surface.

2. The method of claim 1, wherein the intermediate compound undergoes an intramolecular rearrangement in which the second nucleophilic group on the anchor molecule displaces the first nucleophilic group, thus forming a more stable bond between the anchor molecule and the polypeptide.

3. The method of claim 1, wherein the polypeptide comprise a thioester.

4. The method of claim 1, wherein the anchor molecule comprises a 2-aminonucleophile or a 3-aminonucleophile.

5. The method of claim 4, wherein the 2-aminonucleophile is a 2-aminothiol.

6. The method of claim 5, wherein the anchor molecule comprises a structure selected from the group consisting of:

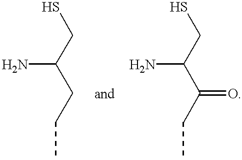

7. The method of claim 1, wherein the anchor molecule is attached to the surface prior to contacting the anchor molecule with the polypeptide.

8. The method of claim 1, wherein the anchor molecule is attached to the surface after contacting the anchor molecule with the polypeptide.

9. The method of claim 1, wherein the anchor molecule comprises a functional group that can be covalently linked to a molecule that is attached to the surface.

10. The method of claim 9, wherein the functional group is selected from the group consisting of ketones, diketones, olefins, epoxides, aldehydes, reactive esters, isocyanates, thioisocyanates, carboxylic acid chlorides, disulfides, sulfonate esters, maleimide, isomaleimide, N-hydroxysuccinimide, nitrilotriacetic acid, activated hydroxyl, haloacetyl, activated carboxyl, hydrazide, epoxy, aziridine, sulfonylchloride, acyl hydrazines, trifluoromethyldiaziridine, pyridyldisulfide, N-acyl-imidazole, imidazolecarbamate, vinylsulfone, succinimidylcarbonate, arylazide, anhydride, diazoacetate, benzophenone, isothiocyanate, isocyanate, imidoester, aminooxy and fluorobenzene.

11. The method of claim 1, wherein the anchor molecule comprises a tag moiety that can be noncovalently bound to a molecule that is attached to the surface.

12. The method of claim 11, wherein the tag comprises a binding domain which is derived from a polypeptide selected from the group consisting of glutathione-S-transferase (GST), maltose-binding protein, chitin, cellulase, thioredoxin, avidin, streptavidin, and green-fluorescent protein (GFP).

13. The method of claim 11, wherein the tag comprises a chitin binding domain or a cellulose binding domain.

14. The method of claim 11, wherein the tag comprises a peptide that comprises an amino-terminal Cys, Thr, or Ser.

15. The method of claim 1, wherein the polypeptide comprises a non-natural amino acid.

16. The method of claim 1, wherein the ester or thioester is chemically introduced onto the polypeptide.

17. The method of claim 1, wherein the ester or thioester is introduced onto the polypeptide by chemical synthesis of the polypeptide.

18. The method of claim 1, wherein the polypeptide that comprises an ester or thioester is obtained by:
expressing a chimeric gene that encodes a fusion protein which comprises:
the polypeptide and an intein, or a functional portion thereof, which is joined to the polypeptide at a splice junction at the amino terminus of the intein, wherein the carboxyl terminus of the intein lacks a functional splice junction; and
contacting the fusion protein with a nucleophilic compound which releases the polypeptide from the intein at the splice junction and forms the polypeptide that comprises a terminal ester or thioester.

19. The method of claim 18, wherein the nucleophilic compound is the anchor molecule.

20. The method of claim 18, wherein the nucleophilic compound comprises a peptide.

21. The method of claim 20, wherein the peptide comprises a serine, threonine or cysteine at its amino terminus, the oxygen and sulfur of which are the nucleophilic groups that undergo the transesterification reaction.

22. The method of claim 18, wherein the nucleophilic compound comprises a thiol as the nucleophile.

23. The method of claim 18, wherein the intein is an Int-n of a split intein and the anchor molecule comprises an amino acid sequence that comprises an Int-c of a split intein, wherein the Int-n and the Int-c undergo an intein splicing reaction, thus attaching the anchor molecule to the polypeptide.

24. The method of claim 23, wherein the Int-n is derived from a dnaE-n gene and the Int-c is derived from a dnaE-c gene.

25. The method of claim 24, wherein the dnaE-n gene and the dnaE-c gene are from a cyanobacterium species.

26. The method of claim 25, wherein the cyanobacterium species is a Synechocystis species.

27. The method of claim 18, wherein the fusion protein is expressed in vitro.

28. The method of claim 18, wherein the fusion protein is expressed in vivo by introducing the chimeric gene into a host cell and incubating the host cell under conditions conducive to expression of the fusion protein.

29. The method of claim 1, wherein the surface comprises a biochip.

30. The method of claim 29, wherein the biochip comprises a non-sample surface and a plurality of sample portions that are elevated with respect to the non-sample surface and each sample portion has attached thereto a single polypeptide species.

31. The method of claim 29, wherein the biochip comprises one or more materials selected from the group consisting of silicon, plastic, gold, and glass.

32. The method of claim 1, wherein the surface comprises a microparticle.

33. The method of claim 1, wherein the polypeptide is placed in contact with the surface using a microvolume dispenser that comprises:
a body; and
at least one vertical channel defined within the body, the channel being defined by at least one passive valve;
wherein an interior surface defining at least one vertical channel is hydrophobic.

34. The method of claim 33, wherein the dispenser comprises a plurality of vertical channels defined within the body.

35. The method of claim 34, wherein the vertical channels are arranged as an array.

36. An array of immobilized polypeptides attached to a surface, wherein the array comprises at least a first polypeptide species and a second polypeptide species and each of which polypeptide species are:
attached to a separate region of the surface;
attached to the surface in the same orientation; and
are folded in a secondary structure as required for a biological activity.

37. The array of claim 36, wherein each of the peptide species are covalently attached to a surface-bound linker by a 2-aminonucleophile ester bond.

38. The array of claim 37, wherein the 2-aminonucleophile ester bond is a 2-aminothioester bond.

39. The array of claim 37, wherein the 2-aminonucleophile ester bond undergoes an intramolecular rearrangement to form an amide bond.

40. The array of claim 37, wherein the linker is a non-peptide linker.

41. The array of claim 36, wherein the C-terminus of each of the polypeptides is attached to the surface.

42. The array of claim 37, wherein the linker comprises a structure selected from the group consisting of:

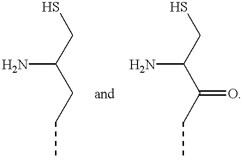

43. The array of claim 36, wherein the surface comprises a biochip.

44. The array of claim 43, wherein the biochip comprises a non-sample surface and a plurality of sample portions that are elevated with respect to the non-sample surface and each sample portion has attached thereto a single polypeptide species.

45. The array of claim 43, wherein the biochip comprises one or more materials selected from the group consisting of silicon, plastic, gold, and glass.

46. An array of immobilized polypeptides attached to a surface which comprises a plurality of surface regions, wherein each surface region has attached thereto a polypeptide species and a polynucleotide that encodes the polypeptide species.

47. The array of claim 46, wherein the surface comprises a biochip.

48. The array of claim 47, wherein the biochip comprises a non-sample surface and a plurality of sample portions that are elevated with respect to the non-sample surface and each sample portion has attached thereto a single polypeptide species and a polynucleotide that encodes the polypeptide species.

49. The array of claim 47, wherein the biochip comprises one or more materials selected from the group consisting of silicon, silicon oxide, plastic and glass.

50. A method for screening a library of nucleic acids to identify a nucleic acid that encodes a polypeptide having a desired activity, the method comprising:
expressing a plurality of fusion proteins, each of which is encoded by an expression cassette that comprises:
a) a member of the library of nucleic acids;
b) an intein coding region; and
c) an open reading frame that encodes a polypeptide that is displayed on a surface of a replicable genetic package;
wherein the fusion proteins are displayed on the surface of a replicable genetic package; and
screening the replicable genetic packages to identify those that display a polypeptide having the desired activity.

51. The method of claim 50, wherein the polypeptide encoded by the library member is released from the fusion protein by contacting the phage with a nucleophilic compound, which nucleophilic compound becomes attached to the polypeptide.

52. The method of claim 51, wherein the nucleophilic compound comprises a compound that has a first nucleophilic group and a second nucleophilic group at a 2 or 3 position relative to the first nucleophilic group.

53. The method of claim 52, wherein the nucleophilic compound is a 2-aminonucleophile or a 3-aminonucleophile.

54. The method of claim 53, wherein the nucleophilic compound is a 2-aminothiol or a 3-aminothiol.

55. The method of claim 51, wherein the nucleophilic compound comprises a thiol or a hydroxyl.

56. A nucleic acid that comprises an expression cassette, wherein the expression cassette comprises:
an insertion site at which a polynucleotide can be introduced into the expression cassette;
an intein coding region, wherein the carboxyl terminus of the intein coding region is mutated so that it does not function as a splice junction for intein-mediated cleavage; and
an open reading frame that encodes a polypeptide that is displayed on a surface of a replicable genetic package;
wherein the introduction of a polynucleotide at the insertion site results in an open reading frame that encodes a fusion protein which comprises a polypeptide encoded by the polynucleotide, which polypeptide is attached at its carboxyl terminus to an amino terminus of the intein, and the surface-displayed polypeptide is attached to a carboxyl terminus of the intein.

57. The nucleic acid of claim 56, wherein the expression cassette further comprises a promoter.

58. The nucleic acid of claim 56, wherein the polynucleotide is a member of a library of polynucleotides.

59. The nucleic acid of claim 58, wherein the library of polynucleotides is a library of cDNA molecules, genomic DNA fragments, or recombination products.

60. A method for immobilizing a polypeptide to a surface, wherein the method comprises:
contacting a polypeptide which comprises an ester or thioester, with an anchor molecule comprising a first nucleophilic group at a 2 or 3 position relative to a second nucleophilic group,
wherein the ester or thioester undergoes a trans-esterification reaction with the first nucleophilic group, thus forming an intermediate compound in which the polypeptide is attached to the anchor molecule through the first nucleophilic group;
wherein said intermediate compound undergoes an intramolecular rearrangement in which the second nucleophilic group on the anchor molecule displaces the first nucleophilic group, thus forming a bond between the anchor molecule and the polypeptide; and
attaching the anchor molecule to a surface.

61. A method for immobilizing a polypeptide to a surface, wherein the method comprises:
contacting a polypeptide which comprises an ester or thioester, with an anchor molecule comprising a reactive group selected from the group consisting of a NH₂—NH—R group and an aminooxy group
wherein R represents an anchor molecule,
wherein the ester or thioester reacts with the reactive group, thus forming a compound comprising a polypeptide attached to the anchor molecule through the reactive group.

62. The method of claim 61, wherein the polypeptide that comprises an ester or a thioester are obtained by:
expressing a chimeric gene that encodes a fusion protein which comprises:
the polypeptide; and
an intein, or a functional portion thereof, which is joined to the polypeptide at a splice junction at the amino terminus of the intein, wherein the carboxyl terminus of the intein lacks a functional splice junction; and
contacting the fusion protein with a nucleophilic compound which releases the polypeptide from the intein at the splice junction and forms the polypeptide that comprises a terminal ester or thioester.

63. The method of claim 62, wherein the nucleophilic compound is the anchor molecule.

64. The method of claim 62, wherein the nucleophilic compound comprises a peptide.

65. The method of claim 64, wherein the peptide comprises a serine, threonine or cysteine at its amino terminus.

66. The method of claim 62, wherein the nucleophilic compound comprises a thiol as the nucleophile.

67. The method of claim 61, wherein the anchor molecule is attached to the surface after contacting the anchor molecule with the polypeptide.

68. The method of claim 61, wherein the anchor molecule comprises a functional group that can be covalently linked to a molecule that is attached to the surface.

69. The method of claim 68, wherein the functional group is selected from the group consisting of ketones, diketones, olefins, epoxides, aldehydes, reactive esters, isocyanates, thioisocyanates, carboxylic acid chlorides, disulfides, sulfonate esters, maleimide, isomaleimide, N-hydroxysuccinimide, nitrilotriacetic acid, activated hydroxyl, haloacetyl, activated carboxyl, hydrazide, epoxy, aziridine, sulfonylchloride, acyl hydrazines, trifluoromethyldiaziridine, pyridyldisulfide, N-acyl-imidazole, imidazolecarbamate, vinylsulfone, succinimidylcarbonate, arylazide, anhydride, diazoacetate, benzophenone, isothiocyanate, isocyanate, imidoester, aminooxy and fluorobenzene.

70. The method of claim 61, wherein the anchor molecule comprises a tag moiety that can be noncovalently bound to a molecule that is attached to the surface.

71. The method of claim 70, wherein the tag comprises a binding domain which is derived from a polypeptide selected from the group consisting of glutathione-S-transferase (GST), maltose-binding protein, chitin, cellulase, thioredoxin, avidin, streptavidin, and green-fluorescent protein (GFP).

72. The method of claim 70, wherein the tag comprises a chitin binding domain or a cellulose binding domain.

73. The method of claim 70, wherein the tag comprises a peptide that comprises an amino-terminal Cys, Thr, or Ser.

74. The method of claim 61, wherein the polypeptide comprises a non-natural amino acid.

75. The method of claim 61, wherein the ester or thioester is chemically introduced onto the polypeptide.

76. The method of claim 61, wherein the ester or thioester is introduced onto the polypeptide by chemical synthesis of the polypeptide.

77. A kit for use in immobilizing one or more polypeptides containing an ester or thioester to a surface of a substrate comprising:
an anchor molecule reagent for adapting said ester or thioester containing polypeptide to said surface,
wherein said anchor molecule comprises a first nucleophilic group at a 2 or 3 position relative to a second nucleophilic group,
wherein the ester or thioester of said one or more polypeptides undergoes a trans-esterification reaction with the first nucleophilic group, thus forming an intermediate compound in which the polypeptides are attached to the anchor molecules through the first nucleophilic group,
wherein said anchor molecule is adapted for attachment to said surface of said substrate.

78. The kit of claim 77 further comprising:
a DNA vector for introducing said ester or thioester into said polypeptide, said vector being adapted to receive a nucleic acid sequence encoding said polypeptide to form a ester or thioester polypeptide expression vector for expressing said polypeptide as an ester or thioester polypeptide having said ester or said thioester incorporated therein.

79. The kit of claim 77 further comprising:
a chemical agent for introducing into said polypeptide an ester or thioester.

80. The kit of claim 77 further comprising:
instructions for instructing a user to carry out the method of claim 1 using said kit.

81. The kit of claim 77 further comprising:
a substrate for attaching said anchor molecules thereto for immobilizing said polypeptides thereon.

82. The kit of claim 81, wherein said anchor molecule is supplied attached to said surface of said substrate for later attaching said polypeptide thereto by a user.

83. The kit of claim 77, wherein said polypeptides are supplied with said kit.

84. The kit of claim 83, wherein said polypeptides are supplied with said kit pre-coupled with said anchor molecule(s).

85. The method of claim 77, wherein said substrate comprises a microparticle.
